(19) United States
(12) Patent Application Publication
(10) Pub. No.: US 2016/0278734 A1
(43) Pub. Date: Sep. 29, 2016

(54) UNIT BLOCK FOR MULTIPLE PURPOSES AND MULTIPLE IMAGES, AND MULTI-MODULE MEDICAL PHANTOM USING UNIT BLOCK

(71) Applicant: KOREA RESEARCH INSTITUTE OF STANDARDS AND SCIENCE, Daejoen (KR)

(72) Inventors: Cheol Pyo Hong, Gangwon-do (KR); Dong Hoon Lee, Daejeon (KR); Bong Yong Ahn, Daejeon (KR); Hyo-Min Cho, Daejeon (KR)

(73) Assignee: Korea Research Institute of Standards and Science, Daejoen (KR)

(21) Appl. No.: 14/369,813

(22) PCT Filed: Sep. 10, 2013
(86) PCT No.: PCT/KR2013/008155

§ 371 Date: Jun. 30, 2014

(51) Int. Cl. A61B 6/00; A61B 6/03

(52) U.S. Cl. CPC A61B 6/583; A61B 6/032; A61B 6/037; A61B 6/481

(57) ABSTRACT

In a medical phantom using a plurality of unit blocks related to an example of the present invention, the plurality of unit blocks includes a first unit block of a hexahedron shape with an empty interior; and a second unit block of a hexahedron shape with an empty interior, having a plurality of ridges formed on a top and a plurality of furrows formed on a bottom to be combined with the plurality of ridges, and the medical phantom is determined according to a combination form of the first unit block and the second unit block.

### TECHNICAL FIELD

The present invention relates to a unit block for multi-purpose multiple images and a multi-module medical phantom using the unit block. Specifically, the present invention relates to a unit block for configuring a phantom and a method of manufacturing the unit block having a form and a structure capable of filling a medium needed for imaging into the unit block and performing image quality evaluation, dose measurement and interventional procedure training.

### BACKGROUND ART

A medical phantom is a model mimicking physical properties of entire or part of a human body, which is used in a variety of forms for a variety of purposes in performing evaluation of performance of a diagnostic and therapeutic device, evaluation of quality of a medical image, dose measurement, interventional procedure training and evaluation and the like.

Particularly, each diagnostic imaging device has different physical mechanism for acquiring an image, and the size and form of the diagnostic imaging device are diverse, and thus phantoms having various forms and properties exist presently.

That is, the medical phantom should be manufactured in a variety of forms to be appropriate to the various sizes and forms of the diagnostic imaging device, and, as a result, the phantom cannot but be restricted by characteristics of the diagnostic imaging device.

Furthermore, in order to make the phantom in a form similar to a human body, it should be manufactured in a size similar to that of the human body, and since a medium is filled therein, the phantom is heavy, and there are a lot of difficulties in operating the phantom.

The human body can be modeled in a variety of ways, and, particularly, when the human body is mimicked, it can be modeled in a combination of voxel units, which are small volume pixels.

In other words, tissues and organs of the human body can be mimicked in various combinations of voxels.

As a result, a phantom may be configured by voxel unit by applying the above concept to the medical phantom, and a phantom using Lego blocks is the best implementation of the concept.

Conventionally, there are some cases in which a phantom of a predetermined form is configured by combining bricks, which are unit blocks of Lego, and using the phantom for performance evaluation of a diagnostic imaging device.

However, in the case of a phantom using Lego blocks, inside of the blocks is an empty space, and thus it is disadvantageous in that the blocks should be imaged after being assembled and contained in a container of a predetermined size.

That is, although the phantom can be assembled in a variety of forms using the blocks, it is disadvantageous in that the blocks should be put into a container having a signal source in order to generate the signal source needed for imaging.

Furthermore, there is a problem in that a medium having a specific physical property is needed inside the blocks for the sake of image evaluation and dose measurement.

As a result, in the case of a phantom using Lego blocks, a degree of freedom of the blocks is restricted by the size and shape of the container, and the phantom has a problem the same as that of a widely used conventional phantom.

In addition, the most serious disadvantage of the Lego blocks is that there are a lot of constraints in the size, shape and functions of the Lego blocks to be used for medical purposes since the Lego blocks are manufactured not for the medical phantom.

In addition, since the Lego-type blocks have ridges and furrows, a combination thereof will be very complicated if the Lego-type blocks are combined and simply imaged, and thus the Lego-type blocks are very disadvantageous in utilizing an image for a medical purpose.

Particularly, since it is disadvantageous in that complexity will be increased extremely if Legos of a small physical size are combined and it is difficult to precisely mimic the characteristics of a human body if the physical size the Lego is relatively large, it is required to provide a solution to this problem.

### DISCLOSURE OF INVENTION

### Technical Problem

Therefore, the present invention has been made in view of the above problems, and it is an object of the present invention to provide a user with a unit block for multi-purpose multiple images, a multi-module medical phantom using the unit block and a control method thereof.

Specifically, an object of the present invention is to provide a unit block for configuring a phantom and details of manufacturing the unit block having a form and a structure capable of filling a medium needed for imaging into the unit block and performing image quality evaluation, dose measurement and interventional procedure training.

Meanwhile, technical problems to be solved in the present invention are not limited to the technical problems described above, and unmentioned other technical problems may be clearly understood by those skilled in the art from the following descriptions.

### Technical Solution

To accomplish the above object, according to one aspect of the present invention, there is provided a medical phantom modeling at least a part of a human body using a plurality of unit blocks, in which the plurality of unit blocks includes: a first unit block of a hexahedron shape with an empty interior; and a second unit block of a hexahedron shape with an empty interior, having a plurality of ridges formed on a top and a plurality of furrows formed on a bottom to be combined with the plurality of ridges, in which the medical phantom is determined according to a combination form of the first unit block and the second unit block, and at least one hole is formed on a side of the first unit block and the second unit block, and a medium may be input through a first hole of the at least one hole, and at least some of the medium and air existing inside the first unit block and the second unit block may be output through a second hole of the at least one hole.

In addition, the plurality of ridges may be formed in a protruded shape on the top of the second unit block, and the plurality of furrows may be formed in a depressed shape on the bottom of the second unit block to be combined with the plurality of ridges, and air may not exist inside the first unit block and the second unit block through the second hole.

In addition, the medium input through the first hole may include H2O needed for magnetic resonance imaging, CuSO4, MnCl2 and NiCl2 based on the H2O, and a Gd series medium, an iron oxide series medium and a gel type medium, which may create a contrast effect.

In addition, the medium input through the first hole may include a material mimicking water, air, bone, contrast media, tissue and/or fat, which can perform an image evaluation in X-ray computed tomography.

In addition, the medium input through the first hole may include positron-emitting isotopes and gamma-emitting isotopes, which are signal sources of a nuclear medicine imaging instrument such as PET and/or SPECT.

In addition, a stem cell corresponding to a tissue of the human body may be provided inside the first unit block and the second unit block.

In addition, the medical phantom determined according to a combination form of the first unit block and the second unit block may be used for multiple purposes and may be connected to a multi-imaging device to support multi-imaging.

In addition, the medical phantom may further include an image quality evaluation module provided inside the first unit block and the second unit block, and the medical phantom may evaluate at least some of spatial resolution, contrast resolution, signal-to-noise ratio, uniformity, position and accuracy of selecting cross-section, and geometric accuracy using the image quality evaluation module.

### Advantageous Effects

The present invention may provide a user with a unit block for multi-purpose multiple images, a multi-module medical phantom using the unit block and a control method thereof.

Specifically, the present invention may provide a unit block for configuring a phantom and details of manufacturing the unit block having a form and a structure capable of filling a medium needed for imaging into the unit block and performing image quality evaluation, dose measurement and interventional procedure training.

Unlike a conventional heavy and hard-to-operate phantom for single image and single purpose, a block-based phantom according to the present invention can be assembled in a variety of forms according to combination of blocks and used for multiple images and multiple purposes, and, particularly, since the unit blocks are very easy to mass-produce and can be used in various imaging devices, it is very economical.

Meanwhile, the effects that can be obtained from the present invention are not limited to the effects described above, and unmentioned other effects may be clearly understood by those skilled in the art from the following descriptions.

### BRIEF DESCRIPTION OF THE DRAWINGS

The above and other features and advantages of example embodiments will become more apparent by describing in detail example embodiments with reference to the attached drawings. The accompanying drawings are intended to depict example embodiments and should not be interpreted to limit the intended scope of the claims. The accompanying drawings are not to be considered as drawn to scale unless explicitly noted.

FIG. 1 is a view showing an example of a phantom for measuring a radiation dose related to the present invention.

FIG. 2 is an exploded perspective view showing an example of the phantom for measuring a radiation dose described in FIG. 1.

FIG. 3 is a view showing various kinds of medical phantoms related to the present invention.

FIG. 4 is a view showing specific examples of different kinds of medical phantoms related to the present invention.

FIG. 5 is a view showing specific examples of a human body modeled in a three-dimensional form in relation to the present invention.

FIG. 6 is a view showing specific examples of a human body modeled using Lego blocks.

FIG. 7 is a view showing other specific examples of a human body modeled using Lego blocks.

FIG. 8 is a view showing specific examples of MRI T2 images obtained by filling water in an oxford block in relation to the present invention.

FIG. 9 is a view showing a basic form of a unit block proposed by the present invention.

FIGS. 10 to 12 are views showing examples of an application form of a unit block proposed by the present invention.

FIG. 13 is a view showing a form of a stem block phantom proposed by the present invention.

FIG. 14 is a view showing a specific form of a stem block phantom applied to multi-purpose multiple images in relation to the present invention.

FIGS. 15 to 17 are views showing specific forms of a QA/AC module applying a stem block phantom applied to multi-purpose multiple images in relation to the present invention.

### BEST MODE FOR CARRYING OUT THE INVENTION

A preferred embodiment of the present invention will be hereafter described in detail, with reference to the accompanying drawings. The embodiment described below does not unduly limit the scope of the present invention, and the entire configuration described in this embodiment may not be said to be prerequisite as a means for solving the problem of the present invention.

A phantom refers to a model used as a substitute in a study on a biomedical system, such as investigating and analyzing distribution of electromagnetic waves in a human body and a specific absorption rate (SAR) of a human tissue.

At this point, quantitative evaluation of the electromagnetic waves received by the human body is performed based on the specific absorption rate, and since it is difficult to actually measure the electromagnetic waves, a so-called phantom identical to a human body is manufactured, and estimation or the like is performed based on measurement of electric fields or temperature increased in the phantom, animal experiments, and electromagnetic field analysis when an electromagnetic wave is radiated.

The phantom needs to have an appearance of a size similar to the structure of a human body tissue and have a relative permittivity ε, a conductivity σ and a density ρ of the human body tissue at each measurement frequency.

Typically, the phantom can be used as a model instead of a human body to determine a radiation dose that the human body receives and may mean an object used to simulate and measure attenuation and scattering of radiation or distribution of radioactive materials in the object.

On the other hand, a medical phantom is a model mimicking physical properties of entire or part of a human body, which is used in a variety of forms and for a variety of purposes in performance evaluation of diagnostic and therapeutic devices, quality evaluation of medical images, dose measurement, interventional procedure training and evaluation and the like.

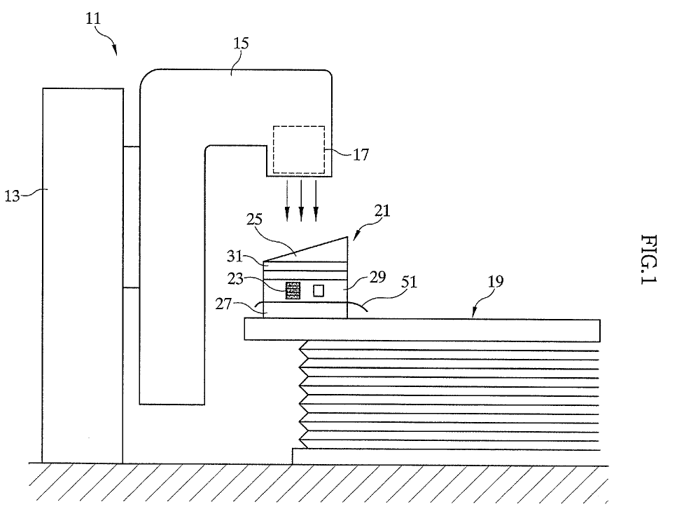

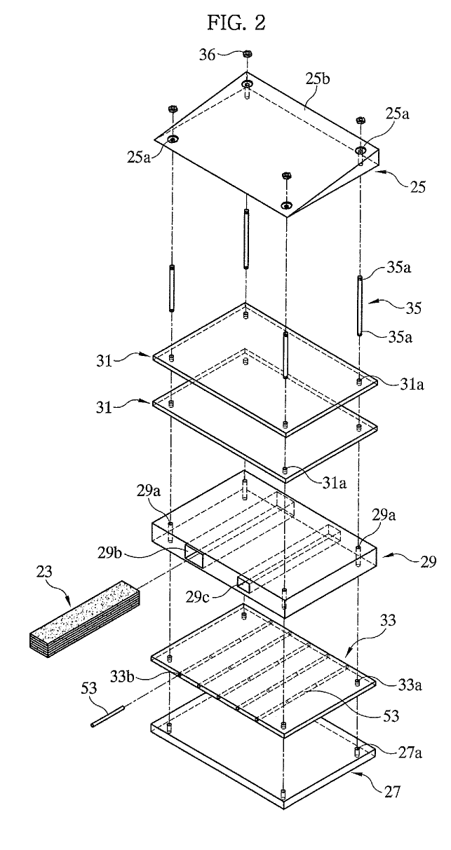

FIG. 1 is a view showing an example of a phantom for measuring a radiation dose related to the present invention, and FIG. 2 is an exploded perspective view showing an example of the phantom for measuring a radiation dose described in FIG. 1.

FIG. 1 is a view showing an example of using a phantom for measuring a radiation dose according to an embodiment of the present invention, and a linear accelerator 11 is described as an example of a radiation generator.

Referring to FIG. 1, it is understood that a phantom according to the present embodiment is placed under a radiation emitting unit 17 in the vertical direction while being put on a treatment table 19. The treatment table 19 is paired with the linear accelerator 11 to make a set, which is a bed on which a patient to be treated lies down.

In addition, the linear accelerator 11 is configured of a main body 13 and a rotation gantry 15 rotatably installed at the main body 13.

A high voltage generator, a microwave generator or the like is installed in the main body 13, and devices such as an acceleration tube for accelerating electrons, a magnetic field generator, the radiation emitting unit 17 and the like are provided inside the rotation gantry 15. Radiation emitted from the radiation emitting unit 17 is radiated on the tumors of the patient lying on the treatment table 19.

Meanwhile, the phantom 21 according to the present embodiment receives the radiation radiated downwardly from the radiation emitting unit 17 while being set under the radiation emitting unit 17 in the vertical direction and may grasp a dose of the irradiated radiation.

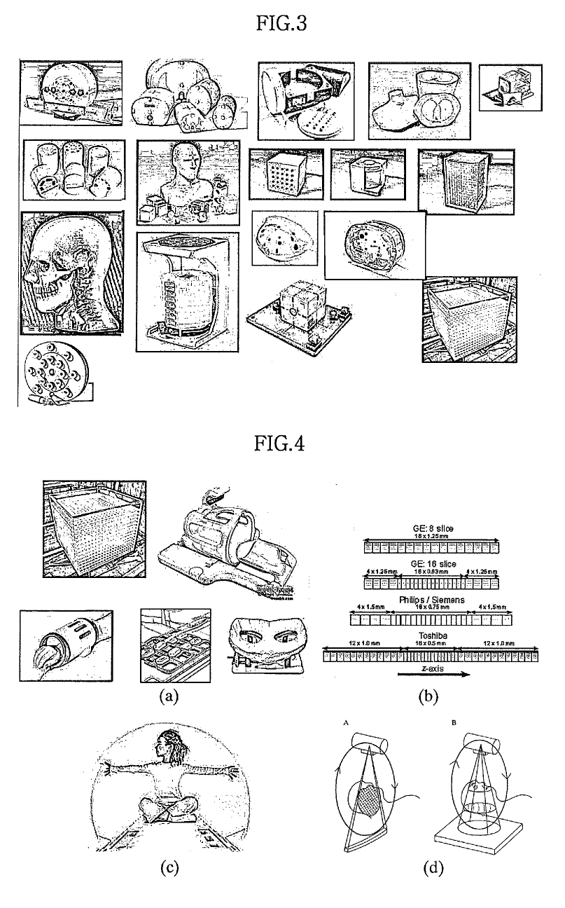

The phantom 21 is configured by combining one or more base plates 27, a mimetic accommodation plate 29 embedded with various kinds of mimetics 23 as needed, a plurality of flat plates 31, a wedge plate 25, a thermoluminescence dosimeter mounting plate (hereinafter, referred to as a TLD mounting plate) (33 of FIG. 2), and an ion chamber mounting plate (39 of FIG. 3). ( [symbol] 39 [symbol] [symbol] )

An example of combining the constitutional components described above may vary without restriction depending on situation and may have, for example, a stacked structure as shown in FIGS. 4 to 6.

Reference numeral 51 is an X-ray film. The X-ray film 51 is a target of radiation passing through the wedge plate 25, the flat plates 31 and the mimetic accommodation plate 29 in order, and an energy level of the radiation arriving at the surface thereof is expressed.

As a result, the X-ray film 51 is a radiation dose measurement unit for measuring (at a corresponding depth) a dose of the radiation radiated from the radiation emitting unit 17. The radiation dose measurement unit further includes the TLD mounting plate 33 and a thermoluminescence dosimeter (hereinafter, referred to as TLD) (53 of FIG. 2) applied thereto, and the ion chamber mounting plate and an ion chamber applied thereto, in addition to the X-ray film 51.

The radiation dose measurement unit has an object of measuring a dose of radiation at a depth where the radiation dose measurement unit is placed.

The depth where the radiation dose measurement unit is placed varies depending on the example of combination described above, and a type of the mimetic 23 placed on the top of the radiation dose measurement unit, whether or not a mimetic is placed or the like varies depending on situation.

FIG. 2 is an exploded perspective view showing a combination example of a phantom for measuring a radiation dose according to an embodiment of the present invention.

As shown in FIG. 2, the phantom 21 for measuring a radiation, dose according to a combination example has a configuration including a base plate 27 having a rectangular plate shape of a predetermined thickness, a TLD mounting plate 33 stacked on the top of the base plate 27, a mimetic accommodation plate 29, a flat plate 31 and a wedge plate 25 stacked on the top of the TLD mounting plate 33 in order, and fixing rods 35 for combining the constitutional components with each other.

As shown in FIG. 1, the base plate 27 horizontally supports the TLD mounting plate 33 at a predetermined height from the treatment table 19 while being laid down on the treatment table 19. The base plate 27 adjusts the distance of the radiation dose measurement unit from the radiation emitting unit 17.

For example, the distance of the radiation dose measurement unit from the radiation emitting unit 17 can be narrowed by manufacturing the base plate 27 to be thick or increasing the number of the base plates 27.

Female screw holes 27a are formed at the four corners of the base plate 27. The female screw hole 27a is a groove formed with female screw threads on the inner periphery thereof, which is combined with a male screw unit 35a formed at the lower end of the fixing rod 35. The fixing rod 35 is vertically extended while being combined with the female screw hole 27a and tightly attaches and fixes the constitutional components.

The TLD mounting plate 33 is a rectangular acryl plate having a predetermined thickness, which has five horizontally extended TLD accommodation holes 33b inside thereof. The TLD accommodation holes 33b have a predetermined diameter and are parallel to each other, and both ends thereof are open to outside. It is apparent that the number of the TLD accommodation holes 33b may vary depending on situation.

For reference, the acryl has a tissue density corresponding to the density of a general tissue of a human body.

TLDs 53 are inserted inside the TLD accommodation holes 33b. As is known to public, the TLD is a dosimeter manufactured using a material having thermoluminescence features, which can be manufactured in the form of a chip or powder. In the case of the powder form, the TLD is tightly sealed in a cylindrical capsule.

In the present embodiment, a capsule-type TLD 53 is used. That is, the capsule-type TLD 53 is inserted into the TLD accommodation hole 33b and then pushed, for example, to the center portion to be placed at a right position. Particularly, a plurality of TLDs 53 can be inserted in one TLD accommodation hole 33b and may be applied only to a selected TLD accommodation hole 33b. The TLD 33 receives radiation radiated from the top while being placed at the TLD accommodation hole 33b and may be collected later by a worker to quantitatively evaluate an exposed radiation dose through a TLD reader (not shown).

The mimetic accommodation plate 29 is provided on the top of the TLD mounting plate 33. The mimetic accommodation plate 29 is a hexahedron acryl block having vertical penetration holes 29a at the four corners and includes two space units 29b and 29c inside thereof.

The space units 29b and 29c are rectangular holes horizontally extended to be parallel to each other, and both ends thereof are open to outside. The cross-sectional shape or size of the space units 29b and 29c may vary without restriction depending on situation.

Basically, the space units 29b and 29c may be left empty or filled with the memetic 23 according to a mimicking target in a human body. For example, when an empty space such as an oral cavity is mimicked, the space unit 29c is left empty. In addition, when a lung is mimicked, a cork known to have a tissue density similar to that of the lung is inserted, and when a bone is mimicked, Teflon having a tissue density similar to that of the bone is inserted. The memetic 23 can be manufactured in the form of a lump of block or may be manufactured in the form of a thin plate to be stacked and used as needed. The mimetic accommodation plate 29 may not be used in some cases.

The flat plate 31 positioned on the top of the mimetic accommodation plate 29 is a rectangular acryl plate having a variety of thicknesses.

The flat plate 31 functions a role of adjusting a distance of a target from the radiation emitting unit 17. Accordingly, the positions or the number of the flat plates 31 used may vary as needed. For example, the flat plate 31 may be placed between the base plate 27 and the TLD mounting plate 33 or, as shown in the figure, may be installed between the wedge plate 25 and the mimetic accommodation plate 29. It is apparent that penetration holes 29a are provided at the four corners of the flat plate 31.

Meanwhile, the wedge plate 25 is an acryl member having side surfaces shaped in a right-angled triangle. The wedge plate 25 has a horizontal bottom surface and a sloped surface 25b inclined at a predetermined angle with respect to the bottom surface. A preferable inclination angle of the sloped surface 25b is fifteen to thirty degrees.

Basically, the wedge plate 25 functions a role of grasping linearly a degree of arrival of radiation on a target having various depths. For example, if radiation is vertically radiated on the wedge plate 25 while an X-ray film is placed under the wedge plate 25, energy of the radiation gradually decreases while the radiation passes through the wedge plate 25 downwardly (thickness of the wedge plate linearly decreases since the wedge plate is inclined), and radiation of the decreased energy is reflected on the X-ray film, and thus information on the attenuation rate of the radiation with respect to the thickness of the acryl can be obtained. The attenuation rate is high as much if the radiation passes through a thick portion of the wedge plate 25, and the attenuation rate is low if the radiation passes through a relatively thin portion.

Penetration holes 25a are also formed at the four corners of the wedge plate 25.

The fixing rods 35 vertically support the constitutional components placed on the top of the base plate 27 (at this point, an example of combining the constitutional components may vary) and pass through the penetration holes 33a, 29a, 31a and 25a of the TLD mounting plate 33, the mimetic accommodation plate 29, the flat plate 31 and the wedge plate 25, and the male screw units 35a formed at the lower ends thereof are fixed to the female screw holes 27a of the base plate 27.

Reference numeral 36 is a nut for tightly attaching the constitutional components to each other by combining the nut with the male screw unit 35a formed at the upper end of the fixing rod 35.

However, although it is described in FIGS. 1 and 2 assuming that the phantom according to the present invention is for measuring radiation, it is apparent that content of the present invention is not limited thereto, but can be applied to a variety of medical purposes.

Meanwhile, FIG. 3 is a view showing various kinds of medical phantoms related to the present invention.

As shown in FIG. 3, each diagnostic imaging device has different physical mechanism for acquiring an image, and the size and form of the diagnostic imaging device are diverse, and thus phantoms having various forms and properties exist presently.

FIG. 4 is a view showing specific examples of different kinds of medical phantoms related to the present invention.

That is, as shown in FIG. 4, the medical phantom should be manufactured in a variety of forms to be appropriate to the various sizes and forms of the diagnostic imaging device, and, as a result, the phantom cannot but be restricted by characteristics of the diagnostic imaging device.

Furthermore, in order to make the phantom in a form similar to a human body, it should be manufactured in a size similar to that of the human body, and since a medium is filled therein, the phantom is heavy, and there are a lot of difficulties in operating the phantom.

The human body can be modeled in a variety of ways, and, particularly, when the human body is mimicked, it can be modeled in a combination of voxel units, which are small volume pixels.

In other words, tissues and organs of the human body can be mimicked in various combinations of voxels.

FIG. 5 is a view showing specific examples of a human body modeled in a three-dimensional form in relation to the present invention.

In a form as shown in FIG. 5, a phantom may be configured by voxel unit by applying the above concept to the medical phantom, and a phantom using Lego blocks is the best implementation of the concept.

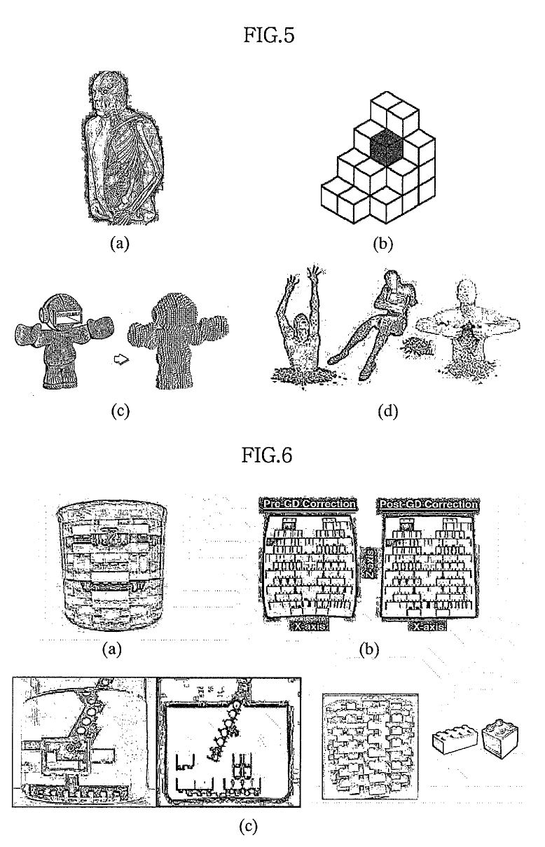

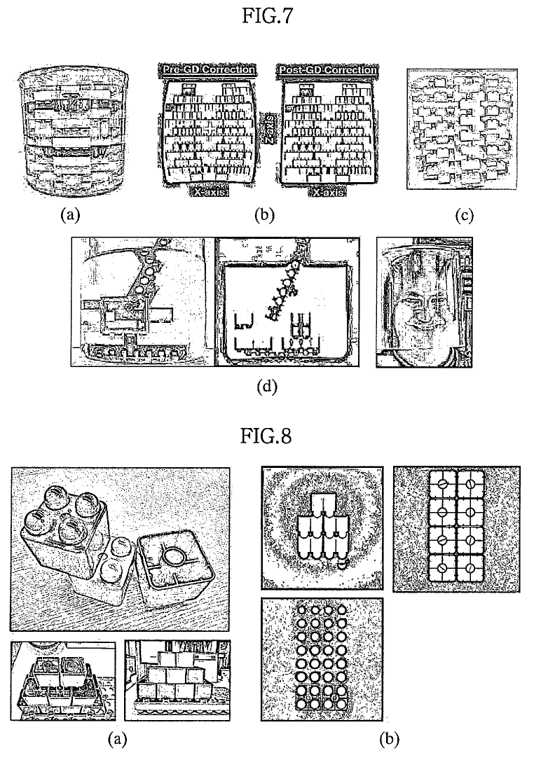

FIG. 6 is a view showing specific examples of a human body modeled using Lego blocks, FIG. 7 is a view showing other specific examples of a human body modeled using Lego blocks, and FIG. 8 is a view showing specific examples of MRI T2 images obtained by filling water in an oxford block in relation to the present invention.

As shown in FIGS. 6 to 8, conventionally, there are some cases in which a phantom of a predetermined form is configured by combining bricks, which are a unit block of Lego, and using the phantom for performance evaluation of a diagnostic imaging device.

However, in the case of a phantom using Lego blocks, inside of the blocks is an empty space, and thus it is disadvantageous in that the blocks should be imaged after being assembled and contained in a container of a predetermined size.

That is, although the phantom can be assembled in a variety of forms using the blocks, it is disadvantageous in that the blocks should be put into a container having a signal source in order to generate the signal source needed for imaging.

Furthermore, there is a problem in that a medium having a specific physical property is needed inside the blocks for the sake of image evaluation and dose measurement.

As a result, in the case of a phantom using Lego blocks, a degree of freedom of the blocks is restricted by the size and shape of the container, and the phantom has a problem the same as that of a widely used conventional phantom.

In addition, the most serious disadvantage of the Lego blocks is that there are a lot of constraints in the size, shape and functions of the Lego blocks to be used for medical purposes since the Lego blocks are manufactured not for the medical phantom.

In addition, since the Lego-type blocks have ridges and furrows, a combination thereof will be very complicated if the Lego-type blocks are combined and simply imaged, and thus the Lego-type blocks are very disadvantageous in utilizing an image for a medical purpose.

Particularly, since it is disadvantageous in that complexity will be increased extremely if Legos of a small physical size are combined and it is difficult to precisely mimic the characteristics of a human body if the physical size the Lego is relatively large.

Accordingly, an object of the present invention is to provide a unit block for multi-purpose multiple images, a multi-module medical phantom using the unit block, and a control method thereof. Specifically, the present invention provides a unit block for configuring a phantom, a method of manufacturing the unit block having a form and a structure capable of filling a medium needed for imaging into the unit block and performing image quality evaluation, dose measurement and interventional procedure training, and a device using them.

That is, the unit block proposed by the present invention may be manufactured in a hexahedron shape so that a medium may be filled inside thereof.

A basic form of the unit block according to the present invention is a unit block configured only in a hexahedron, and an application form may be a structure capable of firmly combining the blocks since there are ridges (male) on the top of the block and furrows (female) on the bottom of the block.

The unit blocks of the basic form and the application form proposed by the present invention may be configured in a variety of forms and sizes by putting together and combining the unit blocks.

At this point, a medium such as CuSO4, MnCl2 or NiCl2, which is a signal source needed for magnetic resonance imaging, or a Gd series medium, an iron oxide series medium or a gel type medium, which may create a contrast effect, may be filled inside the unit block.

In addition, a medium such as water, Iodine, Barium, CaCO3, Paraffin, Adipose or the like, which can perform image evaluation in X-ray computed tomography, or a positron-emitting isotope or a gamma-emitting isotope, which is a signal source of a nuclear medicine imaging instrument such as positron emission tomography (PET) or single photon emission computed tomography (SPECT), may be filled inside the unit block.

Particularly, multi-imaging at a multi-imaging device, as well as single imaging at a single imaging device, can be accomplished through various combinations of the unit blocks.

In addition, the block-based phantom configured as described above may be used for dose measurement in a radiation treatment and temperature measurement in a thermal treatment.

In addition, the medical phantom may further include an image quality evaluation module inside the unit block to evaluate a spatial resolution, a contrast resolution, a signal-to-noise ratio, uniformity, a position and accuracy of selecting a cross-section, geometric accuracy or the like through combination of the unit blocks.

Particularly, it may be supported to acquire even the information on the quality of a photographed area which cannot be imaged by an existing phantom by using the image quality evaluation module together with a phantom used for image quality evaluation.

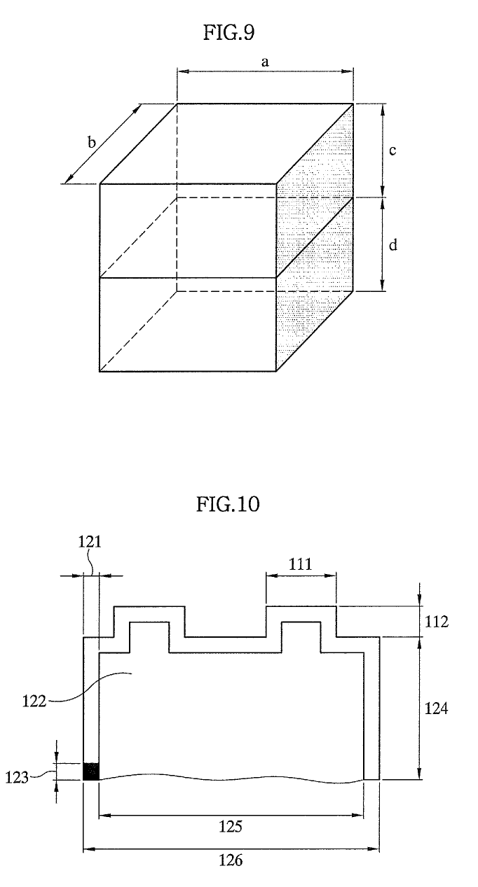

FIG. 9 is a view showing a basic form of a unit block proposed by the present invention.

FIG. 9 is a view showing the configuration of a unit block of a basic form according to the present invention. In FIG. 9, ‘a’ means length of a block (phantom), ‘b’ means width of a block (phantom), ‘c’ means height of a block (phantom), and ‘d’ means length of a block (cap).

As shown in FIG. 9, inside of the unit block according to the present invention is an empty space and formed in a structure which can be tightly sealed after a medium appropriate to the purpose of a medical image is filled inside thereof.

It may be configured such that the side surface of the unit block according to the present invention has two holes, and a medium can be injected through one hole, and the injected medium and the inner air may come out through the other hole, so that air may not be generated at all inside the unit block.

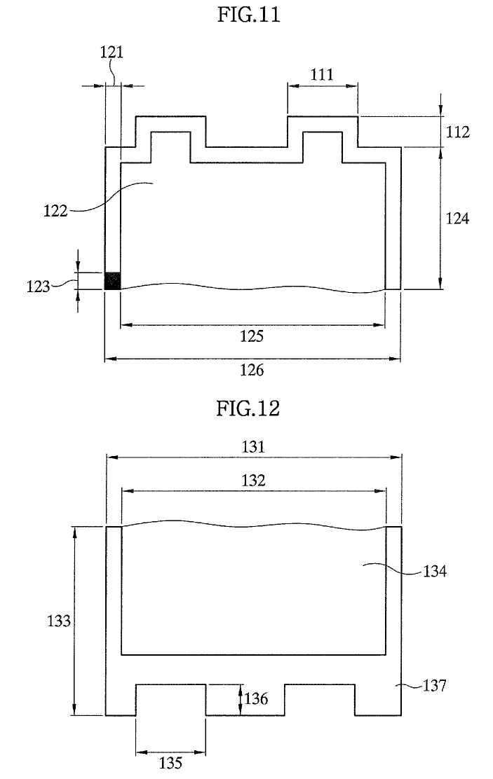

Meanwhile, FIGS. 10 to 12 are views showing examples of an application form of a unit block proposed by the present invention.

Detailed configuration of a unit block of an application form is shown in FIGS. 10 to 12.

In FIGS. 10 to 12, 111 means width of the socket, 112 means height of the socket, 121 means thickness of the container, 122 means fluid of the phantom, 123 means the inlet of phantom fluid, 124 means height of the block (phantom), 125 means inlet width of the block (phantom), and 126 means outlet width of the block (phantom).

In addition, 131 means outlet width of the block (cap), 132 means inlet width of the block (cap), 133 means height of the block (cap), 134 means fluid of the phantom, 135 (→137 ???) means a container material, 136 (→135 ???) means width of the socket, and 137 (→136 ???) means height of the socket.

Referring to FIGS. 10 to 12, since the unit block of the application form is configured of ridges and furrows, unit blocks can be combined with and separated from each other and configured to be combined in a variety of forms.

The ridge parts are formed in a cubic form, and the furrows are configured to accurately attach the cubes of the ridge parts.

In addition, inside of the unit block is an empty space and configured in a structure which can be tightly sealed after a medium appropriate to a purpose is filled inside thereof.

In addition, it is configured such that the side surface of the unit block, other than the ridges and furrows, according to the present invention has two holes, and a medium can be injected through one hole, and the injected medium and the inner air may come out through the other hole, so that air may not be generated at all inside the unit block.

That is, a basic form of the block according to the present invention is a unit block configured only in a hexahedron, and an application form is an application block having ridges (male) on the top and furrows (female) on the bottom, and a structure capable of combining basic unit blocks and blocks of the application form may be configured.

Accordingly, the unit blocks of the basic form and the application form proposed by the present invention may be configured in a variety of forms and sizes by putting and combining the unit blocks together.

At this point, a medium such as CuSO4, MnCl2 or NiCl2, which is a signal source needed for magnetic resonance imaging, or a Gd series medium, an iron oxide series medium or a gel type medium, which may create a contrast effect, may be filled inside the unit block.

In addition, a medium such as water, Iodine, Barium, CaCO3, Paraffin, Adipose or the like, which can perform image evaluation in X-ray computed tomography, or a positron-emitting isotope or a gamma-emitting isotope, which is a signal source of a nuclear medicine imaging instrument such as PET or SPECT, may be filled inside the unit block.

Particularly, multi-imaging at a multi-imaging device, as well as single imaging at a single imaging device, can be accomplished through various combinations of the unit blocks.

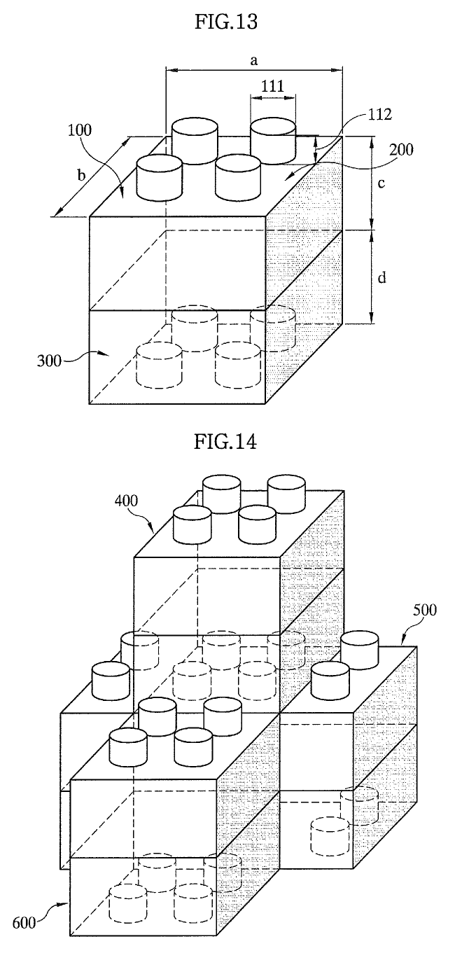

Meanwhile, FIG. 13 is a view showing a form of a stem block phantom proposed by the present invention.

FIG. 13 shows details of filling a medium such as CuSO4, MnCl2 or NiCl2, which is a signal source needed for magnetic resonance imaging, or a Gd series medium, an iron oxide series medium or a gel type medium, which may create a contrast effect, inside the unit block.

Referring to FIG. 13, a medium such as water, Iodine, Barium, CaCO3, Paraffin, Adipose or the like, which can perform image evaluation in X-ray computed tomography, or a positron-emitting isotope or a gamma-emitting isotope, which is a signal source of a nuclear medicine imaging instrument such as PET or SPECT, may be filled inside the unit block.

In addition, FIG. 14 is a view showing a specific form of a stem block phantom applied to multi-purpose multiple images in relation to the present invention.

That is, FIG. 14 shows a basic figure of allowing multi-imaging at a multi-imaging device, as well as single imaging at a single imaging device, through various combinations of the unit blocks.

As shown in FIG. 14, the block-based phantom configured as described above may be used for dose measurement in a radiation treatment and temperature measurement in a thermal treatment, as well as for a multi-imaging device.

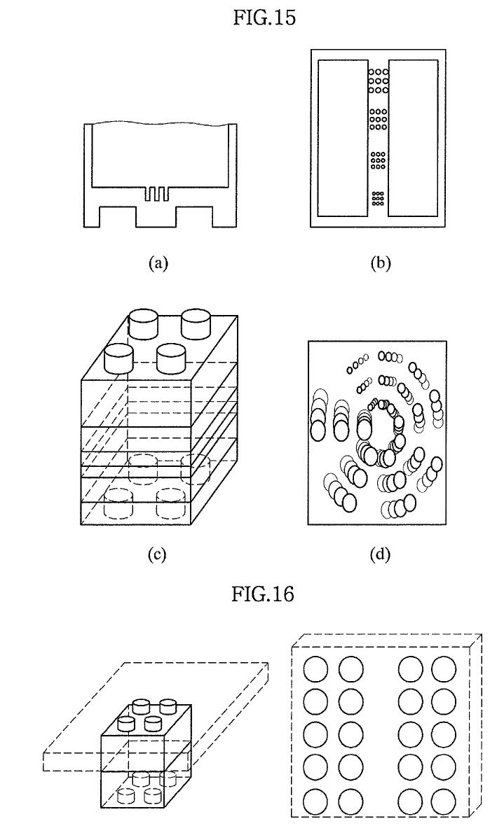

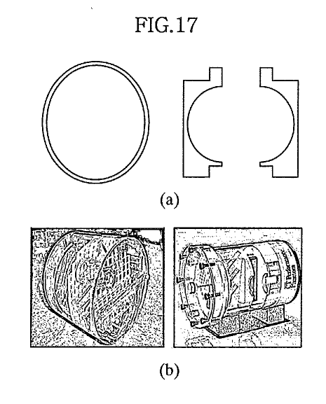

Meanwhile, FIGS. 15 to 17 are views showing specific forms of a QA/AC module applying a stem block phantom applied to multi-purpose multiple images in relation to the present invention.

That is, FIGS. 15 to 17 are views showing module diagrams in which an image quality evaluation module is added inside the unit block to evaluate a spatial resolution, a contrast resolution, a signal-to-noise ratio, uniformity, a position and accuracy of selecting a cross-section, geometric accuracy or the like through combination of the unit blocks.

Referring to FIGS. 15 to 17, even the information on the quality of a photographed area which cannot be imaged by an existing phantom can be acquired by using the image quality evaluation module together with a phantom used for image quality evaluation.

When the configuration of the present invention described above is applied, unlike a conventional heavy and hard-to-operate phantom for single image and single purpose, a block-based phantom can be assembled in a variety of forms according to combination of blocks and used for multiple images and multiple purposes.

Particularly, since the unit blocks are very easy to mass-produce and can be used in various imaging devices, it is very economical.

In addition, since performance evaluation, quality control and dose measurement of an imaging device are very important factors in the medical field and all clinical institutes may use the unit block owing to the characteristics of the block which can be combined in a variety of forms, the block may be widely distributed and used to provide users with high business values and marketability.

Meanwhile, the present invention can be implemented as a computer-readable code in a computer-readable recording medium. The computer-readable recording medium includes all kinds of recording devices for storing data that can be read by a computer system. Examples of the computer-readable recording medium are ROM, RAM, CD-ROM, a magnetic tape, a floppy disk, an optical data storage device and the like, and, in addition, a medium implemented in the form of a carrier wave (e.g., transmission through the Internet) is also included. In addition, the computer-readable recording medium may be distributed in computer systems connected through a network, and a code that can be read by a computer in a distributed manner can be stored and executed therein. In addition, functional programs, codes and code segments for implementing the present invention can be easily inferred by programmers in the art.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

1. A medical phantom, which is a model modeling at least a part of a human body using a plurality of unit blocks,
the plurality of unit blocks includes:
a first unit block of a hexahedron shape with an empty interior; and
a second unit block of a hexahedron shape with an empty interior, having a plurality of ridges formed on a top and a plurality of furrows formed on a bottom to be combined with the plurality of ridges, wherein
the medical phantom is determined according to a combination form of the first unit block and the second unit block,
at least one hole is formed on a side of the first unit block and the second unit block,
a medium is input through a first hole of the at least one hole, and
at least some of the medium and air existing inside the first unit block and the second unit block are output through a second hole of the at least one hole.

2. The medical phantom according to claim 1, wherein
the plurality of ridges is formed in a protruded shape on the top of the second unit block,
the plurality of furrows is formed in a depressed shape on the bottom of the second unit block to be combined with the plurality of ridges, and
air does not exist inside the first unit block and the second unit block through the second hole.

3. The medical phantom according to claim 1, wherein the medium input through the first hole includes H2O needed for magnetic resonance imaging, CuSO4, MnCl2 and NiCl2 based on the H2O, and a Gd series medium, an iron oxide series medium and a gel type medium, which may create a contrast effect.

4. The medical phantom according to claim 1, wherein the medium input through the first hole includes a material mimicking water, air, bone, contrast media, tissue and/or fat, which can perform an image evaluation in X-ray computed tomography.

5. The medical phantom according to claim 1, wherein the medium input through the first hole includes positron-emitting isotopes and gamma-emitting isotopes, which are signal sources of a nuclear medicine imaging instrument such as PET and/or SPECT.

6. The medical phantom according to claim 1, wherein a stem cell corresponding to a tissue of the human body is provided inside the first unit block and the second unit block.

7. The medical phantom according to claim 1, wherein the medical phantom determined according to a combination form of the first unit block and the second unit block can be used for multiple purposes and can be connected to a multi-imaging device to support multi-imaging.

8. The medical phantom according to claim 1, further comprising an image quality evaluation module provided inside the first unit block and the second unit block, wherein
the medical phantom may evaluate at least some of spatial resolution, contrast resolution, signal-to-noise ratio, uniformity, position and accuracy of selecting cross-section, and geometric accuracy using the image quality evaluation module.

9. A diagnostic imaging device using the medical phantom according to claim 1.
